(19) United States
(12) Patent Application Publication
(10) Pub. No.: US 2026/0108368 A1
(43) Pub. Date: Apr. 23, 2026

(54) DEVICE FOR DETERMINING A DISTANCE, METHOD, AND USE

(71) Applicant: Heraeus Medical GmbH, Wehrheim (DE)

(72) Inventors: Michael SCHMITZ, Wehrheim (DE); Harald GRÜN, Messerich (DE); Christian KUNKEL, Marktheidenfeld (DE); Jochen POHLMANN, Marktheidenfeld (DE); Sebastian VOGT, Wehrheim (DE)

(21) Appl. No.: 19/362,951

(22) Filed: Oct. 20, 2025

(30) Foreign Application Priority Data
Oct. 23, 2024 (EP) 24208247.7

(51) Int. Cl. A61F 2/46; A61F 2/30; A61F 2/36

(52) U.S. Cl. CPC A61F 2/4657; A61F 2/30721; A61F 2/30942; A61F 2/3609; A61F 2/3662; A61F 2002/30326; A61F 2002/30354; A61F 2002/3055; A61F 2002/30565; A61F 2002/30583; A61F 2002/30604; A61F 2002/30733; A61F 2002/3611; A61F 2002/3625; A61F 2002/4658

(57) ABSTRACT

A device for determining the required distance between a head region and a shaft region of a joint spacer, to a system, to a method for producing a joint spacer, and to a use. A device for determining the required distance between a head region and a shaft region of a joint spacer includes a head and a shaft, which can be positioned at different distances from one another, wherein the device further includes a fixing apparatus for fixing a distance between the head and the shaft. The device allows the required shape of a hip joint spacer to be determined intraoperatively by adjusting the distance of the head from the shaft to the specific anatomy of the patient.

### CROSS-REFERENCE TO RELATED APPLICATION

This application claims priority pursuant to 35 U.S.C. 119(a) to European Application No. 24208247.7, filed Oct. 23, 2024, which application is incorporated herein by reference in its entirety.

The invention relates to a device for determining a required distance between a head region and a shaft region of a joint spacer, to a system, to a method for producing a joint spacer, and to a use. Preferably, the device is also designed to combine any head with any shaft to obtain a joint spacer that is optimally adapted to the patient's anatomy.

In the context of two-stage replacement operations for endoprostheses, for example hip or shoulder total joint endoprostheses, spacers are used as temporary placeholders in the interim phase. It is used in particular in septic replacement operations. Such spacers are often produced intraoperatively by medical personnel, for example from bone cement such as polymethylmethacrylate bone cement. During the production of these spacers, one or more antibiotics specifically tailored to the germs present can be added to the bone cement, depending on the available antibiogram of the microbial germs causing the infections.

Prefabricated spacers that offer great stability but are not adapted to the individual anatomy of the patient already exist. Therefore, individual spacers are preferably produced intraoperatively.

For the intraoperative production of spacers, usually with bone cement, plastics casting molds are commonly used, as described, for example, in the document U.S. Pat. No. 6,361,731 B1. These casting molds can be produced with different diameters of the spacer head. In this case, the medical user can choose between predefined sizes of the spacer head. In this way, a customized spacer can be provided for a patient depending on the specific anatomical situation. A spacer for a joint or a joint part is called a joint spacer.

In a development, the documents U.S. Pat. Nos. 7,637,729 B2, 7,789,646 B2, 8,480,389 B2 and 8,801,983 B2 propose multi-part casting molds for the production of modular hip spacers. The casting molds from the documents U.S. Pat. Nos. 7,789,646 B2, 8,480,389 B2 and 8,801,983 B2 consist of a casting mold for the shaft, which can be connected to a casting mold for the spacer head. Casting molds for the spacer head with different diameters are available. The shaft casting mold is connected to the spacer head casting mold, which has the selected diameter. The casting mold thus assembled can then be filled with bone cement. After curing, the formed hip joint spacer is removed.

The document EP 3 957 280 B1 describes a device for producing hip joint spacers which makes it possible to produce patient-specific hip joint spacers with regard to the size of the spacer head and the distance of the head from the femoral shaft (femoral offset). This allows further adjustment of the spacer.

The object of the invention is to determine the required shape of a joint spacer simply and reproducibly in order to improve the individually adapted production of joint spacers.

The object is achieved by the device according to claim 1 and by the system, the method and the use according to the coordinated claims. Advantageous embodiments can be found in the dependent claims.

In order to achieve the object, a device is used to determine the required distance between a head region and a shaft region of a joint spacer. The device comprises a head and a shaft that can be positioned at different distances from one another. The device further comprises a fixing apparatus for fixing a distance between the head and the shaft.

The device is designed to be temporarily inserted into the body of a patient in the position of a spacer to be inserted, in order to determine the required distance between the head region of the spacer and the shaft region of the spacer or to determine whether a selected distance fits the specific anatomical conditions. The device is designed to fix the selected distance between the head and the shaft to thereby prevent any unplanned change in the distance or any possible unplanned separation of the shaft and head, which could result in the loss of the head. In particular, the distance is fixed during use in the patient's body. The device can also be called a test gauge.

The device allows the required shape of a hip joint spacer to be determined during surgery by adjusting the distance of the head from the shaft (also known as “femoral offset”) to the specific anatomy of the patient. This means that the correct spacer can be selected individually for each patient. For example, the spacer can then be produced intraoperatively using hip joint spacer molds and/or according to the document EP 3 957 280 B1.

The spacer to be produced and inserted comprises a head region (head) and a shaft region (shaft) and, if necessary, an intermediate neck region (neck) that connects the head region and the shaft region. The spacer, particularly at least in its head region, is modeled on the shape and size of the corresponding bone, for example the femur (thigh bone). The shaft region of the spacer is inserted into the opening in the bone and typically fixed there. The head region of the spacer is approximately spherical, at least in some regions, to reproduce the movable connection, for example with the pelvis, in this case the hip joint. The spacer may further include a neck region connecting the shaft to the head.

The head of the device is also shaped similarly to the head of the corresponding joint, for example the femur. The head may have an approximately spherical outer surface, at least in some regions. The head may be partially or completely hollow. The shaft of the device is designed to be inserted into the opening in the bone. For example, the shaft is elongate and tapers in cross-section to make it easy to insert.

The head and the shaft are typically movable relative to one another, in particular linearly displaceable, for positioning at different distances. The fixation device fixes the head directly or indirectly to the shaft. In the locked (fixed) state, the head and the shaft are connected, if necessary by means of a neck in between, in such a way that at least a relative movement between the head and the shaft which changes the distance is prevented. This can, for example, be a movement in the axial direction, relative to a longitudinal axis of the shaft or a neck located between the shaft and the head. A longitudinal axis means in particular a central longitudinal axis.

In a simple embodiment, the shaft and the head are connected with a thread. For example, the shaft or a neck connected to the shaft has an external thread, and the head has an internal thread. The axial position of the head in relation to the shaft can then be adjusted by relative rotation.

The fixing apparatus can, for example, contain a pin which is designed for example to be retractable in order to be able to fix the position of the head in relation to the shaft. For this purpose, the pin can be moved into a suitable receptacle, for example. Alternatively, or additionally, the fixing apparatus may comprise a locking element which may be pre-tensioned by a spring. In this way, movement between the head and shaft can be blocked when the head is locked and secured with the spring. If the locking element is released against the spring force, for example by manual pressing, a movement between the head and shaft can be released. It can be provided that the movement is possible only when the locking element is pressed.

In one embodiment, the device further comprises a neck connecting the head to the shaft. The neck is in particular firmly connected to the shaft. The neck can be integral with the shaft. In particular, the head is designed to be pushed or screwed onto the neck and/or fixed to the neck.

In particular, the neck is designed so that different distances can be set between the shaft and the head. For example, the head can be arranged in different positions on the neck.

The neck may form an angle with the shaft that is not 180°. In the femur region, the angle can correspond to the CCD angle (caput-collum-diaphyseal angle). The angle may be at least 100°, preferably at least 110°, in particular at least 120° and/or at most 160°, preferably at most 150°, in particular at most 140°. This design enables the production of a particularly well-adapted spacer that is optimally adapted to the patient's anatomy.

In one embodiment, the head on one side and the shaft and/or the neck on the other side are separate from one another or can be separated from one another.

In this embodiment, the shaft and the head can be connected to one another, possibly indirectly via the neck, and then fixed in the desired position relative to one another by means of the fixing apparatus. This makes it possible to exchange the head or the shaft or to choose specifically to use different combinations of shaft and head. For example, a desired head size can be chosen that best fits the patient's anatomy. Alternatively, or additionally, a shaft of a specific length and/or diameter can be selected as best fits the patient's anatomy.

In one embodiment, the head is displaceable on the neck. The fixing apparatus comprises at least one pin and a plurality of form-locking elements. The pin can be brought into contact with one or two form-locking elements in such a way that a displacement of the head on the neck is blocked.

The pin is arranged on one of the two parts that are displaceable relative to one another, in particular on the head. The form-locking elements are arranged on the other of the two parts that are displaceable relative to one another, on the neck. In particular, the head is displaceable on the neck along the axial direction relative to the longitudinal axis of the neck. The longitudinal axis of the neck may correspond to the axis of symmetry and/or the longitudinal axis of the head. In particular, the axial position of the head on the neck is fixed.

The pin is an element that protrudes relative to its surroundings. The pin can have a round or square, for example also elongate or rectangular, cross-section. In principle, the pin can be of any shape. To block the displacement, it is merely necessary for the pin to be able to be brought into contact with the form-locking element in order. In particular, the pin is aligned at least approximately in the radial direction.

The pin can typically be brought into contact with any of the form-locking elements. The form-locking elements are arranged typically in different positions relative to a longitudinal axis of the element having the form-locking elements, in particular in a row. By selecting the form-locking element to be brought into contact with the pin, the relative position and thus the distance between the head and neck is thus fixed. The form-locking elements are aligned in the peripheral direction.

In principle, contact of the pin with one form-locking element is sufficient to block displacement in one direction. Preferably, the pin can be arranged between two form-locking elements to block displacement in both directions. There is a gap in each case between two adjacent form-locking elements, in which the pin can be positioned to fix the distance.

In principle, there are at least three form-locking elements and two gaps in between, so that two different distances can be set depending on the choice of the gap. In particular, at least four or five, or more form-locking elements are present.

In particular, there are two pins that are spaced apart from one another in the axial direction. Typically, the two pins lie on an imaginary line that runs in the axial direction. The axial direction refers to the component that has the pins. In this way, jamming is reliably prevented. There may be two sets of form-locking elements and gaps between them. Each pin can then engage with one set of form-locking elements.

In one embodiment, an axially aligned groove is arranged adjacent to the plurality of form-locking elements. The pin can be displaced in the groove in the axial direction. The distance can thus be set.

In this case, the form-locking elements are in particular comb-shaped. The axially aligned groove adjacent to the form-locking elements is used to displace the head on the neck in particular. The pin, which is arranged in particular on the head, can slide in the groove while the head is displaced axially on the neck. In plan view, the gaps and/or the form-locking elements are arranged perpendicularly to the groove.

In particular, by rotating the head relative to the neck about an axial axis, an engagement can be achieved in which the pin is arranged between two form-locking elements and axial displacement is prevented. A set distance can therefore be fixed after setting the desired distance, by a relative rotation of the head in relation to the neck.

In particular, the groove is arranged with respect to a peripheral surface of the component having the groove in an adjacent angular position of the component to the form-locking elements or their gaps. In particular, the groove is directly connected to the respective gaps, so that upon relative rotation the pin can be moved from the groove into one of the gaps and in this way the distance can be fixed.

In one embodiment, the fixing apparatus comprises a switching apparatus for switching from an open position to a closed position. In particular, the distance between head and shaft can be changed in the open position and/or the distance between head and shaft is fixed in the closed position.

Accordingly, in the open position, a desired distance can be set. Switching can subsequently be performed, to fix the desired distance. In particular, the switching apparatus is also designed to switch from the closed position back to the open position.

Preferably, it is possible to switch back and forth several times between the two positions. In this way, the desired distance can be determined with several iterations.

The switching apparatus can in particular be operated manually. Preferably, the switching apparatus is a mechanical switching apparatus. In this case, no electrical or electronic components are required for switching.

In principle, switching can be effected by moving one component of the device relative to another component of the device, for example by moving a switch relative to a housing part. The movement can be a linear and/or a rotational movement.

Changing the distance between the head and the shaft in the open position does not necessarily have to be possible by simply displacing it. It may be necessary to perform a rotation before the displacement, for example in order to disengage a pin from one or more form-locking elements. Movement between the head and shaft is possible in the open position.

This embodiment prevents unwanted displacement of the head relative to the neck and loosening of the head, for example while testing the possible shape of the spacer with the device in the patient.

In one embodiment, the device comprises a scale for reading a distance between the head and the shaft. The scale can be arranged in the region of form-locking elements, so that the position of a pin indicates a position or a distance on the scale.

In one embodiment, the switching is effected by rotation of a rotating unit relative to a housing part about a longitudinal axis of the neck. A rotating unit is a unit that is mounted so that it can rotate. In particular, switching back to the other position can then be done by rotating again, for example in the opposite direction.

In particular, the neck has a housing. The housing can define the outer shell of the neck. The housing represents a transition between the head, for example different heads, and the shaft, for example different shafts, and can therefore also be called an adapter. In particular, the rotating unit is rotatable relative to the entire housing.

In one embodiment, a locking element is provided which is activated when switching to the closed position and blocks any movement between the head and the shaft. In this embodiment, both an axial displacement and a rotation between the head and neck are blocked in the closed position. Accidental release can also be prevented in this way. Therefore, in the closed position no movement is possible between the head and the shaft.

If the switching is effected by rotation of a rotating unit relative to a housing part and if the rotating unit is rotatable relative to a housing part about a longitudinal axis of the neck, the locking element may be part of the rotating unit. The locking element can thus be easily activated and deactivated by rotation. The locking element is in particular part of the switching apparatus.

In the closed position, the locking element can prevent the pin from moving out of a gap between two form-locking elements in the peripheral direction. This can prevent a rotation of the rotating unit in the neck or the neck housing. The locking element can be an axially extending web. There may be an axial groove or recess in the radial direction next to the web so that the pin can be moved out of the gap in the open position.

There may be a window-like opening, for example in the housing of the neck, through which the locking element can be moved, for example rotated, from a recessed or open position into the closed position.

For example, after setting and fixing a desired distance as described above, the locking element can be activated to secure the desired distance.

In one embodiment, the locking element is connected to a spring-loaded button which interacts with two recesses. The button is positioned in a first recess when the fixing apparatus is in the closed position. The button is positioned in a second recess when the fixing apparatus is in the open position.

The positioning of the button thus indicates the closed position or the open position. The button is located in particular on the rotating unit. The recesses are located in particular in the housing part. The button is typically secured in the recess in such a way, for example by form-locking, that switching is not possible. In particular, rotation of the rotating unit relative to the housing part is not possible because the button is arranged in the recess in such a way that rotation of the rotating unit relative to the housing part is blocked. For switching, the button can be pushed radially inward so that it dips under the material forming the recess against the spring force. In this position, the rotatable part can be rotated relative to the housing part. If the button is then located under the other recess, it is pushed outward in a radial direction by the spring force and thus snaps into the other recess. The device is now in the other position, respectively.

The two recesses are in particular window-like and can therefore also be called windows. The two recesses can be connected. There may therefore be two different, defined regions of a single recess, which are referred to according to the invention as two recesses. In particular, the two recesses are arranged at different angular positions relative to the longitudinal axis. In particular, the two recesses are arranged at the same length position with respect to the longitudinal axis.

The button is part of the locking element and in particular is firmly connected to it. The locking element and button therefore rotate together about the longitudinal axis. In particular, the button is attached to a leaf-shaped spring and is pre-loaded outward. For example, visual symbols can be arranged next to the recesses to indicate the respective position (open position/closed position), so that the current position can be directly seen from the position of the button.

In one embodiment, the fixing apparatus further comprises at least one further pin which, starting from the pin, is in a position rotated by 180° about the longitudinal axis. Alternatively, or additionally, the fixing apparatus further comprises a further plurality of form-locking elements which, starting from the plurality of form-locking elements, are located in a position rotated by 180° about the longitudinal axis.

The fixing against axial movement therefore takes place at two opposite positions. This prevents tilting in relation to the longitudinal axis and ensures secure fixing. There can be two webs and two sets of form-locking elements in each angular position, in order to prevent tilting perpendicularly to the longitudinal axis.

In one embodiment, the device has an outer shell in the region of the head and/or the shaft, which delimits a cavity to the outside. In particular, a filling opening for filling bone cement into the cavity is arranged in the outer shell.

The outer shell is typically not completely hollow. The cavity extends in particular between the outer shell and a core of the head or shaft device, which may be made of metal, for example. The core can serve to stabilize and/or set the device. In particular, the head and/or the shaft is at least partially hollow. The neck can also be partially hollow. The device may contain additional metal reinforcement in the shaft and/or neck.

The filling opening is used for filling bone cement. The filling opening is typically designed to accommodate any filler. Bone cement can then harden inside the device, thus forming a solid and stable spacer.

In particular, the device can serve as a lost mold in this case. The outer shell then remains on the bone cement and, together with this and possibly one or more cores, e.g. made of metal, forms the spacer. This allows a dimensionally stable spacer to be produced directly from the device. Typically, there is at least one filling opening in the head and at least one filling opening in the shaft.

It is particularly preferred that at least the outer shell of the device is made of polymethyl methacrylate. There may be a passage in the end of the neck facing the shaft, through which bone cement that has been filled into the shaft can flow into the interior of the neck.

Typically, at least one vent opening is arranged at least on one side of the respective cavity in the shaft and/or in the head facing away from the filling opening. In this way, upon filling with bone cement the air in the cavity can easily flow out so that no air bubbles remain in the cavity.

In one embodiment, the device comprises a biocompatible material or is made of such a material. For example, biocompatible plastics material can be used. Biocompatible means the property of not having a negative influence on the metabolism of living tissue when in direct contact with it.

In one embodiment, the device may comprise or be made from one or more of the following materials: polycarbonate, polyethylene, polypropylene, polyethylene terephthalate, polybutylene terephthalate, polymethyl methacrylate.

In one embodiment, the device can be sterilized. In particular, the device can be sterilized by means of gamma radiation, electron beam radiation, X-ray radiation and/or ethylene oxide.

A further aspect of the invention is a system comprising a device in particular according to the invention and
at least one additional head that is positionable on the shaft of the device, the head being of a different size from the head of the device, and/or,
at least one additional shaft on which the head of the device can be positioned, the shaft being of a different size from the shaft of the device.

Different sizes of heads refer in particular to their diameter. Different sizes of shafts refer in particular to their axial length and/or diameter.

In particular, there are at least two additional heads, each of different sizes. If necessary, at least two additional shafts in different sizes are available.

In one embodiment, the system includes at least three additional heads and at least three additional shafts. For example, there may be a total of four long shafts and four short shafts. With three or four locking steps for distance adjustment, tens to hundreds of combinations are thus possible. The system can be packaged as a kit and made available in this way to medical personnel for surgery.

A further aspect of the invention is a method for determining a required distance between a head region and a shaft region of a joint spacer, in which a head and a shaft of a device are positioned at a desired distance from one another and the distance between the head and the shaft is fixed. The device may be a device according to the invention. All features, embodiments and advantages of the above-mentioned device can also apply to the method and vice versa.

Another aspect is a method for producing a joint spacer, in which a head and a shaft of a device are positioned at a desired distance from one another, the distance is fixed, and bone cement is filled into the head and/or the shaft.

The device may be a device according to the invention. All features, embodiments and advantages of the above-mentioned device can also apply to the method and vice versa.

First, a required distance is determined by positioning the head and shaft and fixing them. The device itself is then used to produce the joint spacer. The device is used here as a casting mold. The bone cement then hardens in the device. The bone cement is in particular polymethylmethacrylate (PMMA) bone cement.

The method may further comprise one or more of the following steps, in any combination:
connecting the head and neck,
displacing the head on the neck to set the required distance,
locking the displacement by bringing a pin into contact with one or two form-locking elements and/or by rotating the head relative to the neck about a longitudinal axis,
switching from an open position to a closed position in order to fix the distance between the head and the shaft and/or to lock a rotation of the head relative to the neck, in particular by rotating a rotating unit, in particular a button being pushed out of a recess.

In particular, a respective cavity in the head and/or shaft is at least partially filled with bone cement. This is done in particular via a filling opening.

In one embodiment, an outer shell of the device forms an outer shell of the spacer. In other words, after the bone cement has hardened, the outer shell of the device defines the outer shell of the spacer or serves as the outer shell of the spacer. The device is therefore used as a lost formwork to produce the spacer.

A further aspect of the invention is a use of a lost formwork for producing a joint spacer.

In particular, a device for determining a required distance of a head region from a shaft region of a joint spacer is used as a lost formwork for producing the joint spacer. The device may be a device according to the invention.

A lost formwork is a formwork that remains in place after the part to be cast has been produced, and thus becomes part of the produced part.

Embodiments of the invention are also explained in greater detail below with reference to figures. Features of the embodiments can be combined individually or in a plurality of the claimed subjects, unless otherwise indicated. The claimed scope of protection is not limited to the embodiments.

### IN THE DRAWINGS

FIG. 1: is a perspective view of a device;

FIGS. 2 to 4: are steps for using a device;

FIG. 5: is an exploded view of a device;

FIG. 6: is a rotating unit of a device; and,

FIGS. 7 to 9: are steps for using a device.

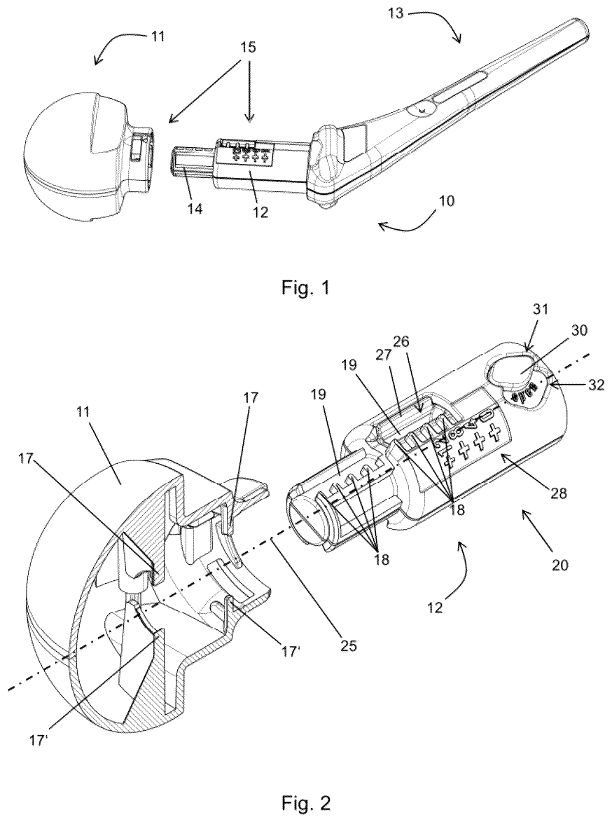

FIG. 1 is a device 10 for determining a required distance between a head region and a shaft region of a joint spacer. A joint spacer 1 is shown by way of example in FIG. 9.

The device 10 comprises a head 11 and a shaft 13. In principle, head 11 and shaft 13 can be arranged at different distances from one another. A neck 12 of the device is fixed at an angle to the shaft 13. The neck 12 may have at its free end an extension 14 pointing away from the shaft 13. The extension 14 may have a reduced diameter compared to the shaft 13. In the example shown here, the head 11 can be placed on the neck 12, in particular on its extension 14. In this case, the extension 14 can be immersed into the head 11 at a desired depth.

Furthermore, the device comprises a fixing apparatus 15 with which the distance between head 11 and shaft 13 can be fixed. In the embodiment shown here, parts of the fixing apparatus 15 are arranged on or in the head 11 and parts of the fixing apparatus 15 are arranged on the neck 12. The fixing apparatus 15 shown here is therefore designed to fix the relative position between head 11 and neck 12.

Details of the fixing by means of the fixing apparatus 15 are shown in FIG. 2 to 4. FIG. 2 is a sectional view of the head 11 and a perspective view of the neck 12. The neck 12 and the head 11 are separate from one another and are arranged on a common axis, namely the longitudinal axis 25 of the neck 12. The head 11 can be pushed along this axis onto the neck 12 and fixed there. A scale 28 is provided, on which the relative position of the head 11 to the neck 12 or the distance between the head 11 and the shaft 13 can be read.

On or in the head 11 there are two pins 17 which are axially spaced from one another. For each pin 17 there is a further pin 17′, which is in particular a copy of the respective pin 17 rotated by 180° about the longitudinal axis 25. On the neck 12 there are two sets of form-locking elements 18. The form-locking elements 18 are designed as parallel webs which run along the peripheral direction of the neck 12, on its surface. Respective gaps are arranged between adjacent form-locking elements 18. A groove 19 extending in the axial direction is arranged peripherally next to the form-locking elements 18, connecting the gaps to one another. On the non-visible underside there are in particular two sets of further form-locking elements 18′. These are each a copy of the respective sets of form-locking elements 18 rotated by 180° about the longitudinal axis 25.

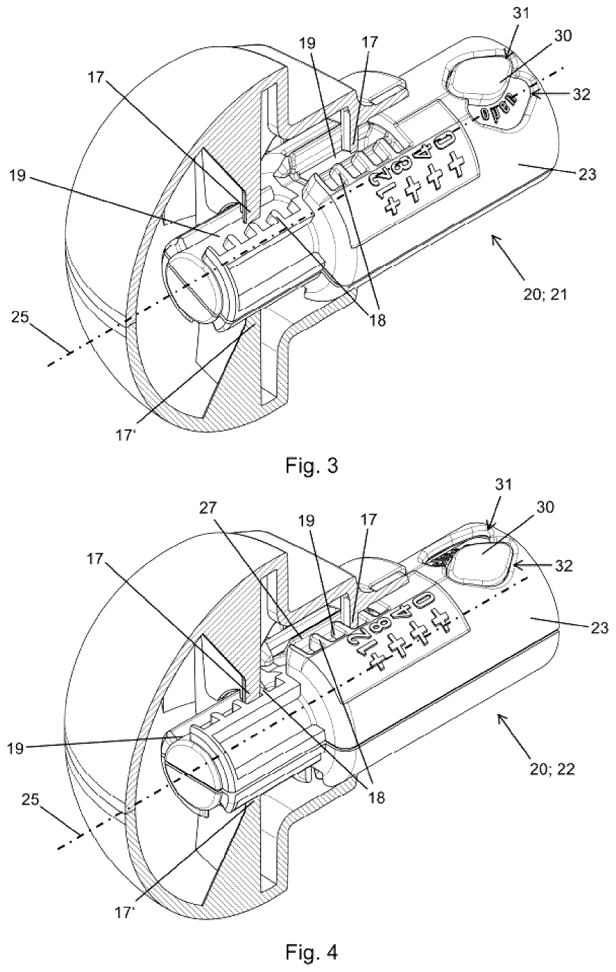

If the head 11 is pushed onto the neck 12, each pin 17 and, if present, each further pin 17′, moves in the axial direction in a groove 19. Such a position is shown in FIG. 3.

If the head 11 is subsequently rotated clockwise relative to the neck 12, the pins 17 and the further pins 17′are moved into one of the gaps and are held against displacement in the axial direction by the adjacent form-locking elements 18 or further form-locking elements 18′. In this way, the distance between head 11 and neck 12 and thus also between head 11 and shaft 13 is fixed.

In order to prevent accidental release of this fixing, a locking element 27 which can be moved into the region of the groove is provided. In this way, a movement of the respective pin 17 or further pin 17′out of the gap into the groove 19 can be prevented. The locking element 37 is part of a switching apparatus 20 which is designed to switch from the open position 21 shown in FIG. 3 to the closed position 22 shown in FIG. 4. In the open position 21, the pin 17 and, if applicable, the further pin 17′ can be moved into the groove 19 by relative rotation between the head 11 and the neck 12 and, as a result, the head 11 can be moved axially with respect to the shaft 13. Thus, the distance between head 11 and shaft 13 can be changed. In the closed position 22, however, the movement of the pin 17 and, if applicable, the further pin 17′, out of the gap is blocked. Thus, any movement between head 11 and shaft 13 is blocked. The distance between head 11 and shaft 13 is fixed.

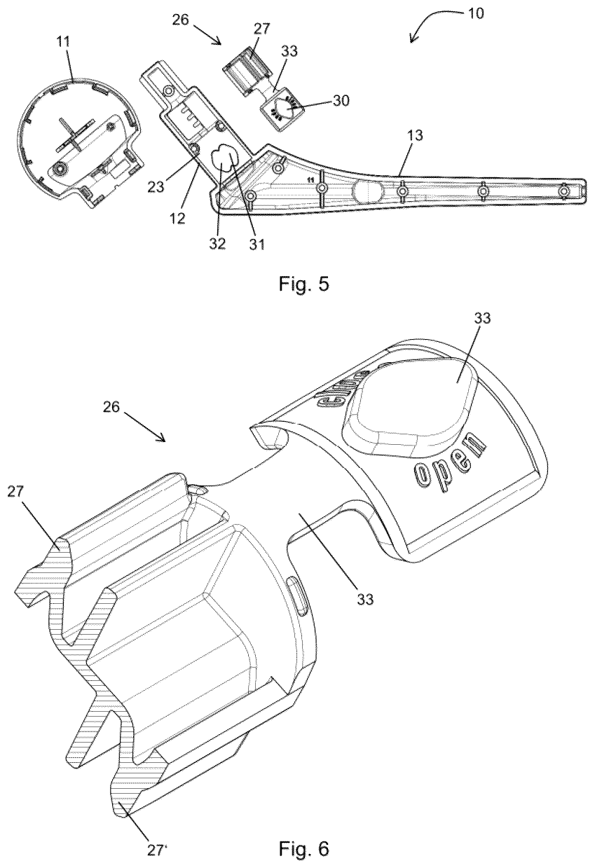

In the embodiment shown here, the locking element 27 is part of a rotating unit 26 which is arranged inside the neck 12 and is rotatable relative to an outer housing part 23 of the neck 12. This is explained in more detail with reference to FIGS. 5 and 6. The rotating unit may comprise a further locking element 27′, which is a copy of the locking element 27 rotated by 180° about the longitudinal axis 25 (see FIG. 6).

FIG. 5 is an exploded view of the device 10 by way of example. In addition to the components head 11, neck 12 and shaft 13 already described the rotating unit 26 can be seen, which is part of the switching apparatus 20 and on which the locking element 17 is located. The rotating unit 26 further comprises a button 30 which is connected to the rest of the rotating unit 26 via a spring element 33. On a housing part 23 of the neck 12, within which the rotating unit 26 is rotatably arranged, there are two recesses 31, 32, each of which is designed to receive the button 30. The recesses 31, 32 are adjacent and connected to one another in the embodiment shown. If the button 30 is arranged in the first recess 31, the device 10 is in the closed position 22. If the button 30 is arranged in the second recess 33, the device 10 is in the open position 21.

The button 30 can be pressed radially inward against the spring force. The button 30 can thus be released from a recess 31, 32. Now the rotating unit 26 can be rotated relative to the housing part 23 until the button 30 reaches the other recess 32, 31 and snaps radially outward into the recess due to the spring force. Due to the arrangement in the respective recess, the respective open position or closed position is fixed, and it is easy to see in which position the fixing apparatus is currently located. For this purpose, corresponding labels are typically provided in the region of the recesses 31, 32 and/or the button 30, as shown by way of example in FIG. 3 to 6.

FIG. 6 is an enlarged, partially sectioned view of the rotating unit 26, in which the button 30 and the spring element 33 can be seen. The spring element is by way of example a leaf-or tongue-shaped part which can be made e.g. of plastics material, for example in one piece with the button and/or the entire rotating unit 26. In the sectional view shown on the left, the radially outwardly projecting locking element 27 and a correspondingly designed further locking element 27′ can be seen.

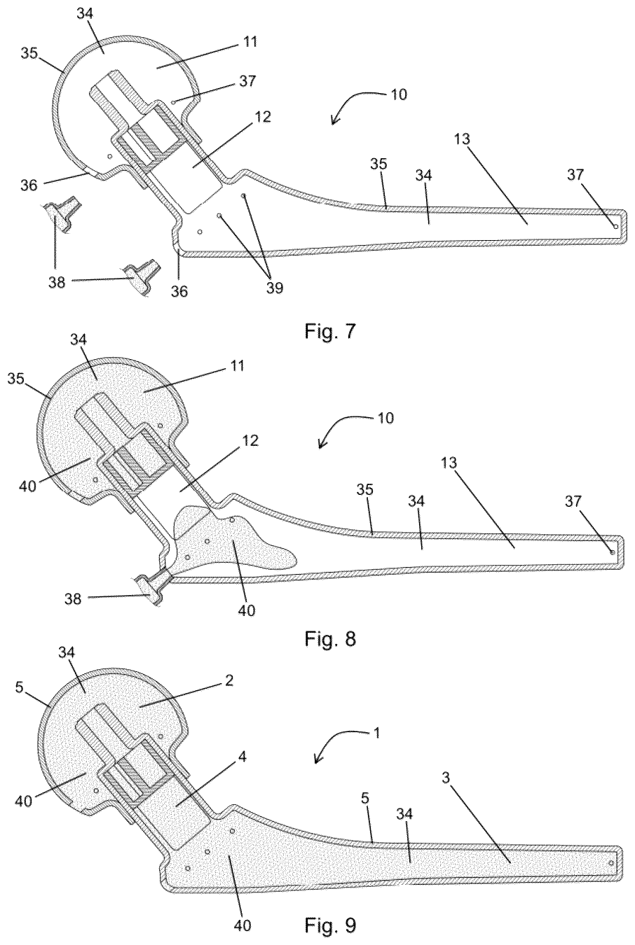

FIG. 7 to 9 are further aspects of the invention. The device 10 according to FIG. 7 comprises an outer shell 35 which delimits an inner cavity 34 toward the outside, specifically in particular both in the region of the head 11 and the shaft 13. In other words, the head 11 and the shaft 13 are at least partially hollow. The outer shell has filling openings 36 through which bone cement or another suitable material can be filled. The device 10 further has vent openings 37 in the outer shell 35. These are preferably arranged so that when filling bone cement 40, for example with one or more applicators 38, as shown in FIG. 8, the displaced air can flow out. A cavity 34 of the neck 12 is connected in particular to a cavity 34 of the head 11 and/or a cavity 34 of the shaft 13 so that bone cement 40 can flow there indirectly via the respective connected part.

FIG. 9 shows the situation after complete filling and hardening of the bone cement 40. The device 10 has now become a joint spacer 1. The joint spacer 1 is individually adapted to the patient's anatomy. The head 11, the neck 12 and the shaft 13 of the device 10 have become a head region 2, a neck region 4 and a shaft region 3 of the joint spacer 1. The joint spacer is produced and consists of the device 10 and the filled and hardened bone cement 40. The outer shell 35 of the device 10 has become the outer shell 5 of the joint spacer 1 and further contains the vent openings 37 and the filling openings 36.

In addition to the existing openings 36, 37, the device 10 or the joint spacer 1 can have outlet openings 39 through which an active substance can pass from the interior of the joint spacer 1 to the outside, to the patient. An active substance, for example comprising one or more antibiotics, can be added to the bone cement 40 before filling, during a septic replacement operation in order to specifically combat the existing germs.

The outlet openings 39 do not have to be different from the vent openings 37; rather, vent openings 37 can also serve as outlet openings 39 and vice versa. However, it is advantageous if outlet openings 39 are provided at several locations, possibly distributed over the entire surface, in particular also at locations where vent openings 37 would be only slightly effective, such as near the filling openings 36

### LIST OF REFERENCE SIGNS

Joint spacer 1
Head region 2
Shaft region 3
Neck region 4
Outer shell 5
Device 10
Head 11
Neck 12
Shaft 13
Extension 14
Fixing apparatus 15
Pin 17
Further pin 17′
Form-locking elements 18
Further form-locking elements 18′
Groove 19
Switching apparatus 20
Open position 21
Closed position 22
Housing part 23
Longitudinal axis 25
Rotating unit 26
Locking element 27
Further locking element 27′
Scale 28
Head 30
First recess 31
Second recess 32
Spring element 33
Cavity 34
Outer shell 35
Filling opening 36
Vent opening 37
Applicator 38
Outlet opening 39
Bone cement 40

What is claimed is:

1. A device for determining a required distance of a head region from a shaft region of a joint spacer, comprising a head and a shaft, which can be positioned at different distances from one another, wherein the device further comprises a fixing apparatus for fixing a distance between the head and the shaft.

2. The device according to claim 1, wherein the device further comprises a neck connecting the head to the shaft.

3. The device according to claim 1, wherein the head on one side and the shaft and/or the neck on the other side are separate from one another or can be separated from one another.

4. The device according to claim 1, wherein the head is displaceable on the neck, the fixing apparatus comprising at least one pin and a plurality of form-locking elements, the pin being able to be brought into contact with one or two form-locking elements in such a way that a displacement of the head on the neck is blocked.

5. The device according to claim 1, wherein an axially aligned groove is arranged adjacent to the plurality of form-locking elements, the pin being able to be displaced in the groove in the axial direction in order to adjust the distance.

6. The device according to claim 1, wherein the fixing apparatus comprises a switching apparatus for switching from an open position to a closed position, it being possible for the distance between the head and the shaft to be changed in the open position and the distance between the head and the shaft being fixed in the closed position.

7. The device according to claim 1, wherein the switching is carried out by rotation of a rotating unit relative to a housing part about a longitudinal axis of the neck.

8. The device according to claim 1, wherein a locking element is provided which is activated when switching to the closed position and blocks any movement between the head and the shaft.

9. The device according to claim 1, wherein the locking element is connected to a spring-mounted button which cooperates with two recesses, the button being positioned in a first recess when the fixing apparatus is in the closed position, the button being positioned in a second recess when the fixing apparatus is in the open position.

10. The device according to claim 1, wherein the fixing apparatus further comprises at least one further pin which, starting from the pin, is in a position rotated by 180° about the longitudinal axis, and in that the fixing apparatus further comprises a further plurality of form-locking elements which, starting from the plurality of form-locking elements, are in a position rotated by 180° about the longitudinal axis.

11. The device according to claim 1, wherein the device comprises an outer shell in the region of the head and/or the shaft, which outer shell delimits a cavity toward the outside, a filling opening for filling bone cement into the cavity being arranged in the outer shell.

12. The system comprising a device according to claim 1 and
at least one additional head which can be positioned on the shaft of the device, wherein the head is of a different size from the head of the device, and/or,
at least one additional shaft on which the head of the device can be positioned, wherein the shaft is of a different size from the shaft of the device.

13. A method for producing a joint spacer, in which a head and a shaft of a device are positioned at a desired distance from one another, the distance is fixed, and bone cement is filled into the head and/or the shaft.

14. The method according to claim 1, wherein an outer shell of the device forms an outer shell of the joint spacer.

15. A method of use of a lost formwork for producing the joint spacer.

16. Device according to claim 2, wherein the head on one side and the shaft and/or the neck on the other side are separate from one another or can be separated from one another.

17. Device according to claim 2, wherein the head is displaceable on the neck, the fixing apparatus comprising at least one pin and a plurality of form-locking elements, the pin being able to be brought into contact with one or two form-locking elements in such a way that a displacement of the head on the neck is blocked.

18. Device according to claim 3, wherein the head is displaceable on the neck, the fixing apparatus comprising at least one pin and a plurality of form-locking elements, the pin being able to be brought into contact with one or two form-locking elements in such a way that a displacement of the head on the neck is blocked.

19. Device according to claim 2, wherein an axially aligned groove is arranged adjacent to the plurality of form-locking elements, the pin being able to be displaced in the groove in the axial direction in order to adjust the distance.

20. Device according to claim 3, wherein an axially aligned groove is arranged adjacent to the plurality of form-locking elements, the pin being able to be displaced in the groove in the axial direction in order to adjust the distance.
